(19) United States
(12) Patent Application Publication
(10) Pub. No.: US 2016/0296293 A1
(43) Pub. Date: Oct. 13, 2016

(54) APPARATUS FOR ROBOTIC SURGERY

(71) Applicant: RENISHAW PLC, Wotton-under-Edge, Gloucestershire (GB)

(72) Inventors: Steven Streatfield GILL, Bristol (GB); Geoffrey MCFARLAND, Wickwar (GB); Robert Neil HARRISON, Quedgeley (GB); Paul David FIELDER, Stroud (GB)

(73) Assignee: RENISHAW PLC, Wotton-under-Edge, Gloucestershire (GB)

(21) Appl. No.: 15/038,544

(22) PCT Filed: Nov. 21, 2014
(86) PCT No.: PCT/GB2014/053445

§ 371 Date: May 23, 2016

(30) Foreign Application Priority Data
Nov. 22, 2013 (GB) 1320618.0

(51) Int. Cl. A61B 34/30

(52) U.S. Cl. CPC A61B 34/30; A61B 2034/302; A61B 2034/2065

(57) ABSTRACT

Surgical apparatus is described that includes a surgical robot having a moveable arm for carrying a surgical tool. The apparatus also has x-ray imaging apparatus that includes at least one of an x-ray source and an x-ray detector. The moveable arm of the surgical robot is configured to carry at least one of the x-ray source and the x-ray detector. In this manner, the surgical robot can be used to perform x-ray imaging to allow patient registration. Methods of using the apparatus are also described.

The present invention relates to surgical robots and in particular to a surgical robot that is also arranged to perform x-ray imaging and a method of using such a robot.

### BACKGROUND

Robots for performing neurosurgical procedures are known. An example of such a robot is the Neuromate (registered trademark) robot manufactured and sold by Renishaw Mayfield SA. An alternative surgical robotic system is described in US2009/0088634.

A neurosurgeon undertaking a surgical procedure (e.g. catheter or electrode implantation in the brain) using a surgical robot such as the Neuromate robot has to firstly register the robot to the patient. In other words, the position of the patient in the local coordinate system of the robot (herein termed the robot coordinate system) needs to be established to allow the robot to drive the surgical instruments (catheters, electrodes etc) to the required target within the patient's brain.

The Neuromate robot is presently registered to the patient by attaching an acoustic transmitter array to a skull mount inserted in the skull bone of the patient and attaching a microphone array to the moveable arm of the robot. The position and orientation of the skull mount can then be found in the robot coordinate system. Previously acquired medical images (CT, MRI images etc) taken with appropriate fiducial markers also attached to the same skull mount can then be registered to the robot. In other words, positions (e.g. target sites, trajectories etc) can be mapped into the local coordinate system of the surgical robot after the patient to robot registration process.

WO2012/085511 describes an alternative registration process that uses an in-theatre CT scanner. The CT scanner is arranged to image a patient's head. A set of x-ray visible fiducial markers are held by the moveable arm of the surgical robot and placed adjacent the patient's head in the field of view of the CT scanner. The position of the x-ray visible fiducial markers held by the arm of the surgical robot (which have a known position in the robot coordinate system) can then be related to positions in the CT image of the patient. Although this technique allows the patient to be registered to the robot, it is time consuming and relies on the positional accuracy of the images acquired using the CT scanner.

### SUMMARY OF THE INVENTION

According to a first aspect of the present invention, there is provided surgical apparatus comprising; a surgical robot having a moveable arm for carrying a surgical tool, and x-ray imaging apparatus including at least one of an x-ray source and an x-ray detector, wherein the moveable arm of the surgical robot is configured to carry at least one of the x-ray source and the x-ray detector.

The present invention thus relates to apparatus including a surgical robot (e.g. a neurosurgical robot) combined with x-ray imaging apparatus. In particular, the moveable arm of the surgical robot not only carries surgical tools for performing surgical procedures on a subject, but also carries an x-ray source or an x-ray detector that can be used to acquire x-ray images of a subject.

Apparatus of the present invention, in which the moveable arm of the surgical robot can also perform an x-ray imaging function, has a number of advantages. As explained above, the first step in any surgical robot based procedure is to “register” the robot to the subject. The registration process involves establishing the position of the relevant part of the subject's anatomy (e.g. the position of a target position within the brain that has been identified by a pre-operative medical scan) in the coordinate system of the surgical robot. Once registered, the surgical robot can then drive instruments (e.g. neurosurgical catheters, shunts, electrodes etc) to the pre-identified target position. The accuracy of a surgical robot based procedure is thus governed not only by the mechanical accuracy of the surgical robot, but also by the accuracy with which the robot is registered to the subject.

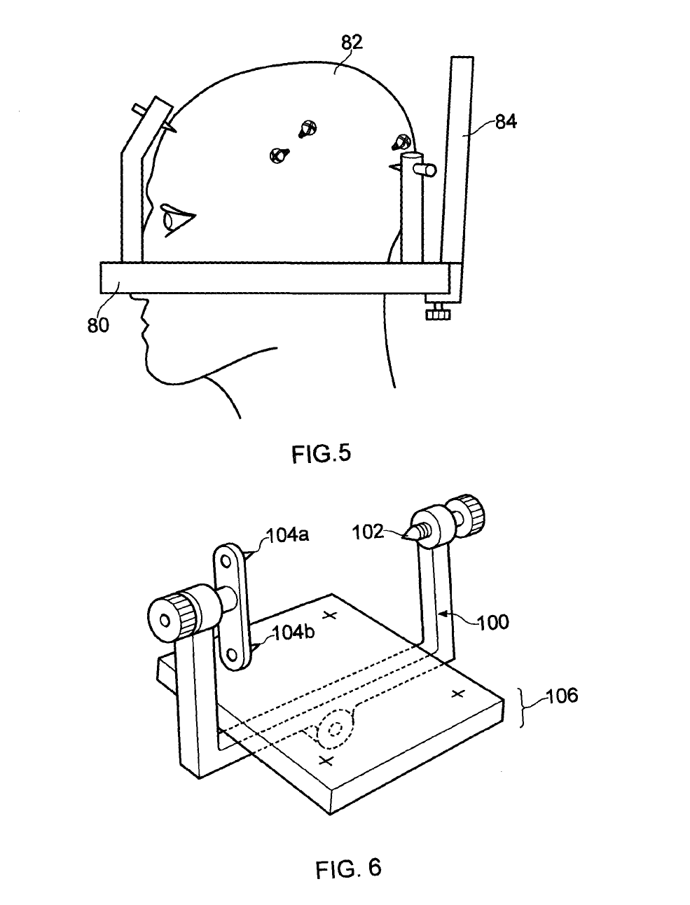

It has been found that surgeons implementing prior art registration techniques for surgical robots, such as the technique described with reference to FIG. 5 in WO2012/085511, can spend a significant amount of time in the operating theatre ensuring that the registration of the surgical robot to the subject is performed with the necessary accuracy. In contrast, the present invention allows x-ray images to be acquired with the x-ray source and/or the x-ray detector held by the moveable arm of the robot in one or more locations that are known to the robot (e.g. in locations that are known in the local coordinate system of the surgical robot). This allows the location of features (e.g. x-ray visible fiducial markers) within acquired x-ray images to be directly established in the robot co-ordinate system without the introduction of any errors that may arise from registering the coordinate system of a totally separate intra-operative x-ray imaging system (e.g. a C-arm or O-arm) with the robot coordinate system. Potential problems associated with ensuring there is no unintended relative movement between the surgical robot and a separate x-ray imaging system during use are also overcome. Furthermore, surgical robots typically have a higher positional accuracy than intra-operative CT scanners thereby allowing higher accuracy position data to be extracted from the acquired x-ray images.

The present invention thus improves the speed and accuracy of registration, meaning that it also becomes practical to adjust the position of the patient during the surgical procedure because the registration procedure can be quickly and reliably performed again. Avoiding the need for a separate CT scanner also reduces the amount of kit that is required in the operating theatre, thereby reducing clutter and improving the surgeon's access to the subject.

The x-ray imaging apparatus may comprise one, or more than one, x-ray source. The x-ray imaging apparatus may comprise one, or more than one, x-ray detector. X-ray imaging requires both an x-ray source and an x-ray detector, but the x-ray imaging apparatus of the present invention may be used with an associated x-ray source and/or an associated x-ray detector as necessary. The x-ray imaging apparatus may comprise an x-ray detector and the moveable arm may be configured to carry the x-ray detector. For example, the x-ray detector may be permanently integrated into the moveable arm or it may be releasably attachable to the moveable arm. The position of the x-ray detector is preferably known (e.g. pre-calibrated) relative to a reference point on the distal end of the moveable arm. In this manner, the position of the x-ray detector is known in the robot coordinate system.

Preferably, the x-ray imaging apparatus comprises an x-ray source and the moveable arm is configured to carry the x-ray source. For example, the x-ray source may be permanently integrated into the moveable arm or it may be releasably attachable to the moveable arm. Conveniently, the moveable arm of the surgical robot is arranged to move and/or re-orientate the x-ray source. For example, the x-ray source may be moved into a plurality of different positions and/or orientations relative to the subject being imaged. This allows a plurality of x-ray images of the subject to be acquired from a plurality of different perspectives. As explained in more detail below, x-ray visible fiducial markers may be attached to a subject being imaged. The x-ray visible fiducial markers may comprise bone screws, spherical balls or the like that are direct attached to bone and/or they may be provided as part of a frame or support (e.g. a head clamp) that is temporarily attached to the subject. The relative position of some or all of the x-ray visible fiducial markers may be known (e.g. from a pre-operative CT scan). As explained in more detail below, the acquisition of a plurality of x-ray images that show x-ray visible fiducial markers from a plurality of different perspectives allow the positions of such markers to be established in the robot coordinate system. This, in turn, allows pre-operatively acquired images (e.g. MRI, CT or CTA scans) that also show the fiducial markers to be mapped across into the robot coordinate system. In this manner, the position of targets identified in images acquired during the pre-operative planning process can be established in the robot coordinate system. The x-ray imaging apparatus may be used only for such registration purposes (e.g. it conveniently may not be used for diagnostic imaging purposes).

The x-ray source of the x-ray imaging apparatus may comprise a medically approved x-ray source. The x-ray source may emit x-rays of an energy greater than 5 KeV, greater than 10 KeV, greater than 20 KeV, or greater than 30 KeV. The strength may be less than 200 KeV, less than 100 KeV, less than 50 KeV, less than 30 KeV or less than 20 KeV. A source strength in the range of 30-100 KeV may conveniently be used. A lower strength source could be used if only fiducial marker location (i.e. not diagnostic imaging) is required. For example, a 5-15 KeV source (e.g. a 10 KeV source) could be used.

The moveable arm of the surgical robot is used to carry both a surgical tool and at least one of the x-ray source and the x-ray detector. The moveable arm may carry both a surgical tool and at least one of the x-ray source and the x-ray detector at the same time. Preferably, the surgical robot comprises a releasable attachment mechanism that allows at least one of the x-ray source and the x-ray detector to be attached to the moveable arm when x-ray imaging is to be performed. Preferably, the releasable attachment mechanism allows the x-ray source and/or the x-ray detector to be attached to the distal end of the moveable arm. It is preferred that the releasable attachment mechanism allows the x-ray source and/or the x-ray detector to be attached to the moveable arm in a repeatable location. The x-ray source and/or the x-ray detector may then be attached to the moveable arm in a repeatable (known) position relative to a reference point at the distal end of the moveable arm. In this manner, the x-ray source and/or the x-ray detector may be removed from and attached to the moveable arm as required. The surgical robot may thus be adapted to perform x-ray imaging as and when needed without its use in surgery being hindered.

The releasable attachment mechanism may be a bespoke linkage for attaching the x-ray source and/or the x-ray detector to the moveable arm of the surgical robot. Advantageous, a tool holder is provided at the distal end of the moveable arm for retaining a surgical tool in a repeatable position. In particular, the tool holder may allow tools to be retained in a repeatable (known) position relative to a reference point at the distal end of the moveable arm. Conveniently, the tool holder also provides the releasable attachment mechanism for attaching at least one of the x-ray source and the x-ray detector to the moveable arm. In other word, an x-ray source or x-ray detector may be attached to the tool holder as required.

Advantageously, the x-ray imaging apparatus comprises at least one x-ray detector. Each x-ray detector preferably comprises a digital x-ray plate. The x-ray plate may be a wired plate or a wireless plate. The digital x-ray plate may support the DICOM image format. As described above, multiple x-ray images of a subject may be acquired from a number of different perspectives by moving the x-ray source. In such an example, acquisition of an image requires an x-ray detector to be positioned to detect at least part of the x-ray beam emitted by the x-ray source. To provide the different perspectives, an x-ray detector may be irradiated from different angles, an x-ray detector may be moved into a plurality of different locations (preferably with knowledge of the relative position of the different locations) and/or multiple x-ray detectors may be placed in different locations (preferably with knowledge of the positional difference between the different locations). One or more x-ray detectors may thus be provided in fixed positions. Alternatively or additionally, at least one x-ray detector may be provided that is moveable between a plurality of different locations. Preferably, the x-ray detector may be placed in any one of a plurality of repeatable locations.

The position of the x-ray detector relative to the associated x-ray source is preferably known or measured. For example, if the x-ray source is carried by the moveable arm of the surgical robot then the location of the x-ray detector is preferably determined in the coordinate system of the robot. This may be achieved by mechanically linking the surgical robot to the x-ray detector such that the x-ray detector adopts a predetermined (known) position relative to the surgical robot (i.e. so the position of the x-ray detector is known in the robot coordinate system).

Advantageous, the at least one x-ray detector comprises a fiducial marker unit located adjacent the digital x-ray plate. Preferably, the fiducial marker unit comprises a plurality of x-ray visible fiducial markers having a known position relative to the digital x-ray plate. In a preferred embodiment, the fiducial marker unit comprises an x-ray transparent sheet (e.g. a Perspex plate) with a plurality of x-ray visible fiducial markers embedded in it or formed on it. The x-ray transparent sheet may have a uniform thickness. Advantageously, a periphery of the x-ray transparent sheet may have increased thickness compared with the central region. A concave central region may be provided to receive a patient's head, whereas the x-ray visible fiducial markers may be located on the peripheral region of increased thickness. The x-ray visible fiducial markers may comprise structured, x-ray visible features (e.g. crosses or other shapes) that cast a shadow onto the digital x-ray plate. The x-ray visible fiducial markers may be located adjacent the edges of the digital x-ray plate, for example near the corners of the digital x-ray plate. The x-ray transparent sheet may then be retained in front of the digital x-ray plate. Information on the location of x-ray source relative to the x-ray detector can then be established (or checked) from the position of the image of the x-ray visible fiducial markers on the digital x-ray plate. If the position of the x-ray source is known (e.g. in the robot coordinate system), the position of the x-ray detector can then be calculated (e.g. in the robot coordinate system). This allows the x-ray detector to be located when required. The calculation of the x-ray detector position relative to the x-ray source may be performed with or without the subject being imaged present.

As mentioned above, if the moveable arm carries an x-ray source then one or more x-ray detectors may be placed at any suitable location. For example, an x-ray detector may be attached to a frame (e.g. a head frame or head clamp) attached to the subject. The x-ray detector may be permanently or releasably attached to the frame. The apparatus of the present invention may thus comprise a frame for attachment to a subject. Advantageously, the frame comprises a head clamp attachable to the skull of a subject. An attachment mechanism may be provided for attaching an x-ray detector to the frame. The x-ray detector may be attachable to the frame in a plurality of different positions or orientations. Preferably, the attachment mechanism allows the x-ray detector to be attached to the frame in only one or in at least one repeatable position.

The apparatus may further comprise a surgical bed or table. The surgical bed may retain a frame (e.g. a head clamp) as described above. The surgical table may include one or more x-ray detectors. The x-ray detectors may be permanently or releasably attached to the surgical bed. The surgical table may include an x-ray detector embedded in the table top. The surgical table may include one or more x-ray detectors that can be attached to the table in one or more locations. For example, x-ray detectors may be slid or slipped into locations on the bed. X-ray detectors may also be provided that flip up from the bed. Preferably, the one or more x-ray detectors may be attached to the frame in one or more repeatable positions.

As described above, the position of an x-ray detector may be known (e.g. by calibration) in the robot coordinate system. It is also described above how the position of an x-ray detector in the robot coordinate system can be measured using one or more x-ray visible fiducial markers that have a known position relative to the x-ray detector. It should, however, be noted that it is not always necessary to establish the position of the x-ray detector. In particular, one or more x-ray visible fiducial markers may be provided that have a known position in the robot coordinate system. For example, one or more x-ray visible fiducial markers may be fitted to a head frame or the like that have a known (calibrated) position in the robot coordinate system. The x-ray detector may then be used to measure the position of targets relative to the x-ray visible fiducial markers without having to establish the position of the x-ray detector in the robot coordinate system.

Alternatively, the moveable arm of the surgical robot may be configured to hold one or more x-ray visible fiducial markers. For example, there may be one or more x-ray visible fiducial markers removeably attached to the moveable arm. Each x-ray visible fiducial marker may be attached to the moveable arm in a repeatable position; e.g. each x-ray visible fiducial marker may have a known (e.g. calibrated) position relative to a datum point on the moveable arm. The moveable arm may also carry an x-ray source and the one or more x-ray visible fiducial markers may be located between the x-ray source and an x-ray detector. In this manner, the x-ray visible fiducial markers visible in the image acquired by the x-ray detector have known positions in the robot coordinate system. It is then possible to establish the position of other features present in x-ray images acquired by the x-ray detector relative to the known positions of the x-ray visible fiducial markers. Again, this process does not require any knowledge of the position of the x-ray detector in the robot coordinate system. A further advantage of this arrangement is that the need for patient mounted fiducials can be removed.

The surgical robot may be of any known type. In a preferred embodiment, the surgical robot may comprise a Neuromate (registered trademark) robot manufactured by Renishaw Mayfield. Advantageously, the surgical robot comprises only the single moveable arm that can carry a surgical tool and at least one of the x-ray source and the x-ray detector. Alternatively, the robot may include one or more additional moveable arms.

The moveable arm of the surgical robot preferably comprises a plurality of motorised joints. For example, a plurality of motorised rotary joints may be provided. The moveable arm may thus be an articulated arm having one or more articulated joints. The moveable arm may also include linear (slider) joints or other movable linkages. Some of the joints of the moveable arm may be motorised, whilst some may be manually actuated. The moveable arm preferably comprises a plurality of arm sections that are linked by a plurality of joints. For example, three or more arm sections may be provided. The proximal end of the moveable arm may be attached to a robot base. The robot base may comprise a floor stand.

Advantageously, the surgical robot comprises measurement means (e.g. a plurality of position encoders) for measuring the position of a reference point at the distal end of the moveable arm in the coordinate system of the surgical robot (i.e. in the so-called robot coordinate system). The position of the reference point in the robot coordinate system can then be tracked as the distal end of the moveable arm is moved about in space. The coordinate system of the surgical robot may be fixed relative to the base of the surgical robot; e.g. the origin of the robot coordinate system may have a fixed position relative to the base of the surgical robot. The position of an x-ray source, x-ray detector and/or tool held by the moveable arm may be known relative to the reference point at the distal end of the moveable arm.

The surgical robot is preferably operated under computer control. For example, movement of the moveable arm may be controlled by a computer. The computer that controls motion of the robot may also be interfaced to the x-ray source and/or x-ray detector of the x-ray imaging apparatus. In this manner, movement of the x-ray source and/or x-ray detector, activation of the x-ray source and collection of x-ray images may be controlled by the same computer. The computer may also perform x-ray image analysis; e.g. it may implement the analyser that is described below.

Advantageously, the apparatus of the present invention comprises an analyser for analysing one or more x-ray images acquired by the x-ray imaging apparatus. The analyser may be arranged to analyse a plurality of x-ray images acquired from different perspectives to establish the position (e.g. in three dimensions) of features within those images. As mentioned above, the apparatus may include one or more x-ray visible fiducial markers that are located within the region being imaged. These x-ray visible fiducial markers may be attached (directly or indirectly) to a subject being imaged and/or may be provided as part of an x-ray detector (e.g. in front of a digital x-ray plate). Preferably, the analyser is arranged to analyse the one or more x-ray images to determine the position of the x-ray visible fiducial markers in a coordinate system of the surgical robot.

The one or more x-ray visible fiducial markers may comprise a plurality of fiducial markers attachable to a subject. Conveniently, the one or more x-ray visible fiducial markers comprise a plurality of bone attachable fiducial markers for direct attachment to a bone of a subject. The bone attachable fiducial markers may comprise bone screws that can be screwed into the bone (e.g. the skull bone) of a subject. The bone attachable fiducial markers may comprise balls or the like that can be inserted into recesses formed in the bone. Advantageously, at least three bone attachable fiducial markers are provided for attachment to each subject. Once attached to the bone of a subject, the relative position of the bone attachable fiducial markers may be measured using different apparatus to the apparatus of the invention. For example, a CT scan may be performed pre-operatively to accurately establish the relative position of the bone attachable fiducial markers. The CT scan, optionally in combination with an MM or CTA scan, may also be used to establish the position of target sites (e.g. within the brain) relative to the fiducial markers. The information on the relative position of the bone attachable fiducial markers may then be used by the analyser of the present invention when calculating the position of the bone attachable fiducial markers from the x-ray images acquired using the apparatus of the present invention. In this manner, positions (e.g. target regions and trajectories) identified in pre-operatively acquired data during surgical planning may be registered to the robot coordinate system from the positions defined by the bone attachable fiducial markers. Although the attachment of fiducial markers to the subject is described above, natural anatomical features or landmarks of the subject (e.g. blood vessel features) may be used to provide one or more x-ray visible fiducial markers.

The apparatus may also include a clamp or frame for attachment to a subject. Advantageously, a head clamp for attachment to the head of a subject is provided. A plurality of x-ray visible fiducial markers may be attached to the clamp or frame. For example, a head clamp may be provided with one or more x-ray visible fiducial markers and/or one or more x-ray visible fiducial markers may be attachable to the head clamp. The relative positions of the x-ray visible fiducial markers provided on the clamp or frame may be known (e.g. they may be located in a known or pre-calibrated relative position). The information on the relative position of the fiducial markers may then be used by the analyser of the present invention when calculating the position of the fiducial markers from x-ray images acquired using the apparatus of the present invention. The measured position of the one or more x-ray visible fiducial markers provided on the clamp or frame may be used to establish the position of the clamp or frame in the robot coordinate system. The subject may be imaged (e.g. using a CT or MM scanner) prior to surgery with the clamp or frame attached thereby allowing positions (e.g. target regions and trajectories) identified in pre-operatively acquired data to be registered to the robot coordinate system. The x-ray visible fiducial markers provided on the clamp or frame may be instead of, or in addition to, the bone attachable fiducial markers mentioned above.

As explained above, the apparatus preferably includes one or more x-ray visible fiducial markers and an analyser that is arranged to analyse one or more acquired x-ray images to determine the position of the x-ray visible fiducial markers in a coordinate system of the surgical robot. A plurality of x-ray images may be acquired with the x-ray source and/or x-ray detector in different positions to allow x-ray images of the subject to be taken from different perspectives. Analysis of such a plurality of x-ray images by the analyser allows the position of the position of the x-ray visible fiducial markers to be measured in three dimensions in the robot coordinate system. Advantageously, at least some of the plurality of x-ray visible fiducial markers have a predetermined positional relationship. For example, the relative position of at least some of the x-ray visible fiducial markers may be predetermined using, for example, a pre-operative (CT or MM) scan. The additional information about the relative position of at least some of the x-ray visible fiducial markers introduces known values in the mathematical analysis such that the accuracy of fiducial position information can be increased and/or fewer x-ray images are required to obtain position information with a certain accuracy.

The surgical robot may be arranged for any type of surgery. Advantageously, the apparatus is arranged for neurosurgery. For example, the surgical robot may be configured for surgery on the central nervous system (e.g. the brain or spinal column) of a human or animal subject. The surgical robot may thus comprise a neurosurgical robot. The surgical tool held by the robot may be of any conventional type. For example, the surgical tool may comprise a surgical instrument (e.g. something that surgically acts on the body) or it may comprise a device (e.g. a guidance device) that allows surgical instruments (e.g. catheters, electrodes etc) to be inserted into the body. In a preferred embodiment, neurosurgical apparatus is provided that allows instruments (e.g. electrodes or catheters) to be accurately guided to target sites within the brain.

The apparatus may include one or more additional features. For example, if an x-ray source is provided (e.g. attached to the moveable arm of the surgical robot) there may also be visible target indicators to show a user where the x-ray beam is directed. For example, a laser may be used to generate a pattern (e.g. crosshairs) that allows the user to perform a visual check of the direction of x-ray emitted by the x-ray source. Such an optical system may also be used to provide macro-alignment of the x-ray source to a subject or an x-ray detector plate.

The apparatus may also include position markers that can be used with in-theatre navigation systems. For example, position markers may be provided at the end of the moveable arm, on the x-ray source, x-ray detector, surgical bed, clamp or frame etc.

According to a further aspect of the invention, there is provided a kit for adapting a surgical robot to perform x-ray imaging, the surgical robot comprising a moveable arm for carrying a surgical tool, wherein the kit comprises an x-ray source and at least one x-ray detector, wherein the x-ray source or the x-ray detector is adapted to be carried by the moveable arm. For example, the moveable arm may include a tool holder. The x-ray source and/or the x-ray detector may be configured so that it can be held by the tool holder.

According to a further aspect of the invention, there is provided a method of operating a surgical robot having a moveable arm for carrying a surgical tool, wherein the method comprises the step of using the moveable arm to carry at least one of an x-ray source and an x-ray detector. The method may include the steps of using the robot to move at least one of an x-ray source and an x-ray detector to acquire one or more x-ray images of a patient and using such images to establish the position of the patient in the robot coordinate system. In other words, the method may provide robot-patient registration.

According to a further aspect of the invention, there is provided an x-ray detector comprising a digital x-ray plate and a fiducial marker unit, wherein the fiducial marker unit comprises a plurality of x-ray visible fiducial markers that have a known position relative to the digital x-ray plate. As explained above, an image of the x-ray visible fiducial markers formed on the digital x-ray plate can then be used to calculate the relative position or positions of the x-ray source and x-ray detector. In a preferred embodiment, the fiducial marker unit comprises an x-ray transparent sheet (e.g. a Perspex plate) with a plurality of x-ray visible fiducial markers embedded in it or on it.

According to a further aspect of the invention, there is provided surgical apparatus comprising a surgical robot having a moveable arm for retaining a surgical tool, and x-ray imaging apparatus comprising an x-ray source and an x-ray detector, wherein the moveable arm is arranged to retain at least one of the x-ray source and the x-ray detector.

According to a further aspect of the invention, there is provided a surgical robot having a moveable arm for retaining a surgical tool, wherein the moveable arm carries an x-ray source. The x-ray source may be integrated into the moveable arm or releasably attachable to the moveable arm.

### DETAILED DESCRIPTION OF THE INVENTION

The invention will now be described, by way of example only, with reference to the accompanying drawings in which;

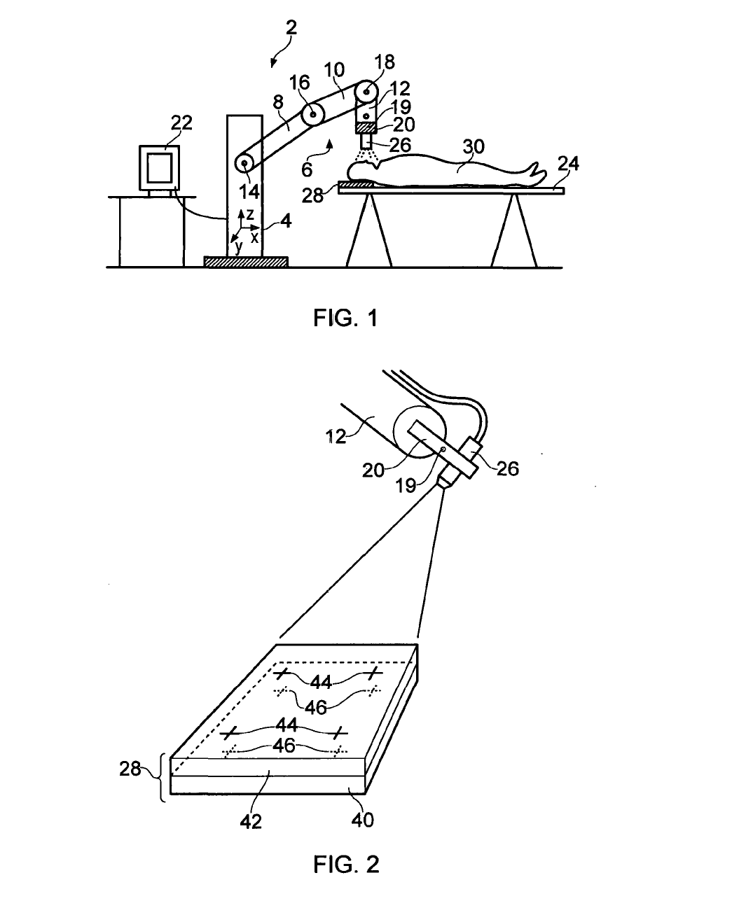

FIG. 1 illustrates apparatus of the present invention,

FIG. 2 illustrates an x-ray source held by a moveable arm of a surgical robot adjacent an x-ray detector,

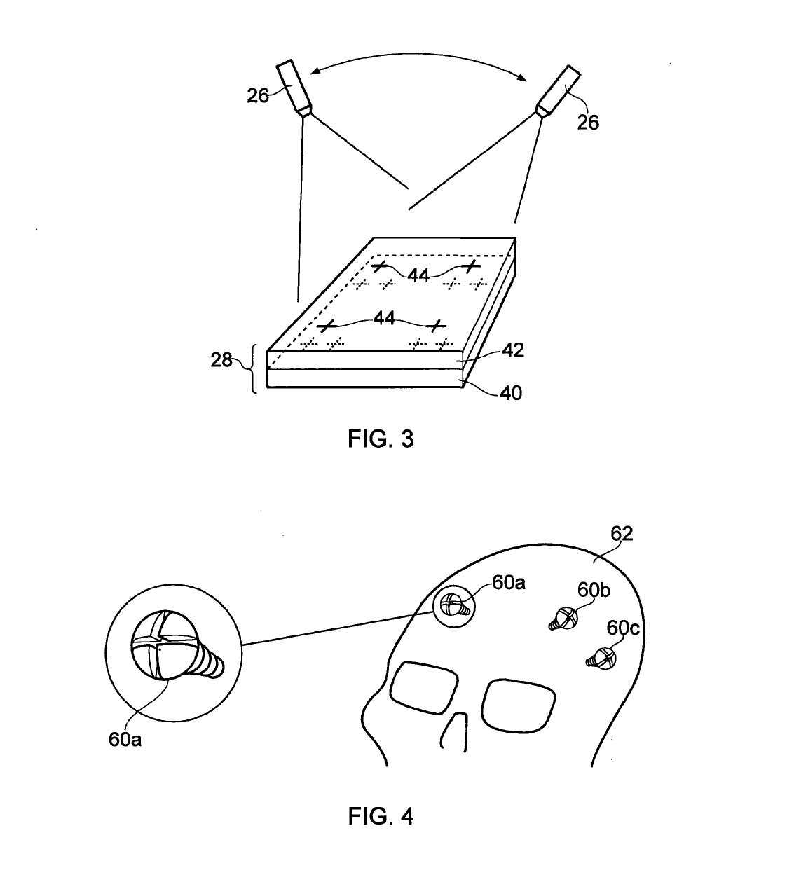

FIG. 3 illustrates how an x-ray source may be directed onto an x-ray detector from a plurality of directions,

FIG. 4 illustrates the attachment of bone screws to the skull of a patient,

FIG. 5 illustrates a head frame with an x-ray detector attached,

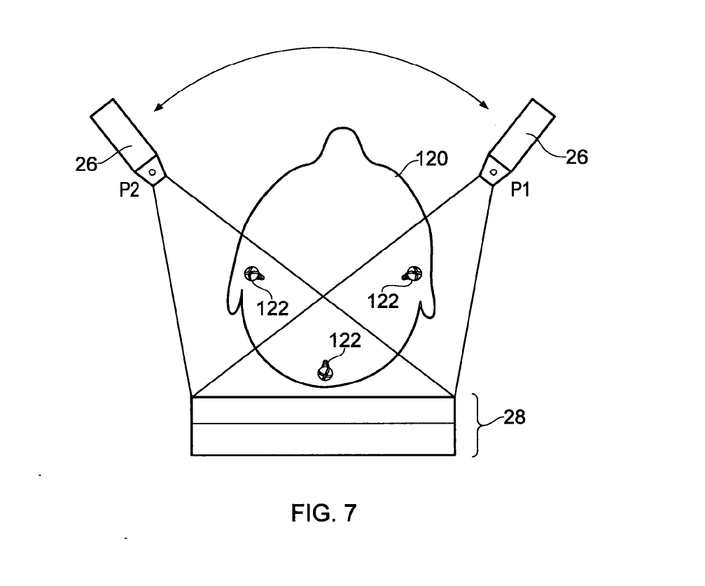

FIG. 6 illustrates a head clamp with an x-ray detector attached,

FIG. 7 shows the acquisition of multiple x-ray images of a patient with attached x-ray visible fiducial markers from two different perspectives,

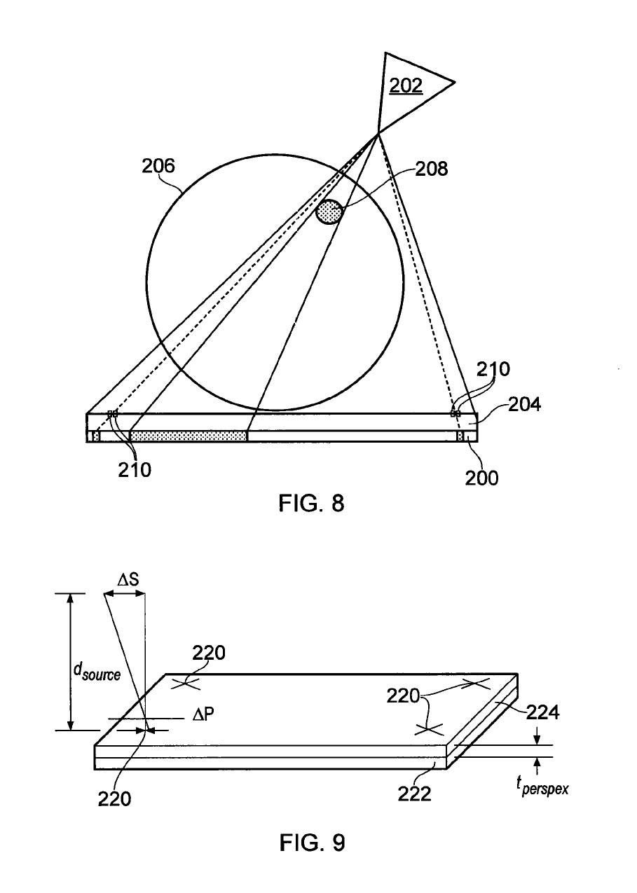

FIG. 8 shows the geometry of an x-ray model,

FIG. 9 shows the Perspex plate and detector arrangement,

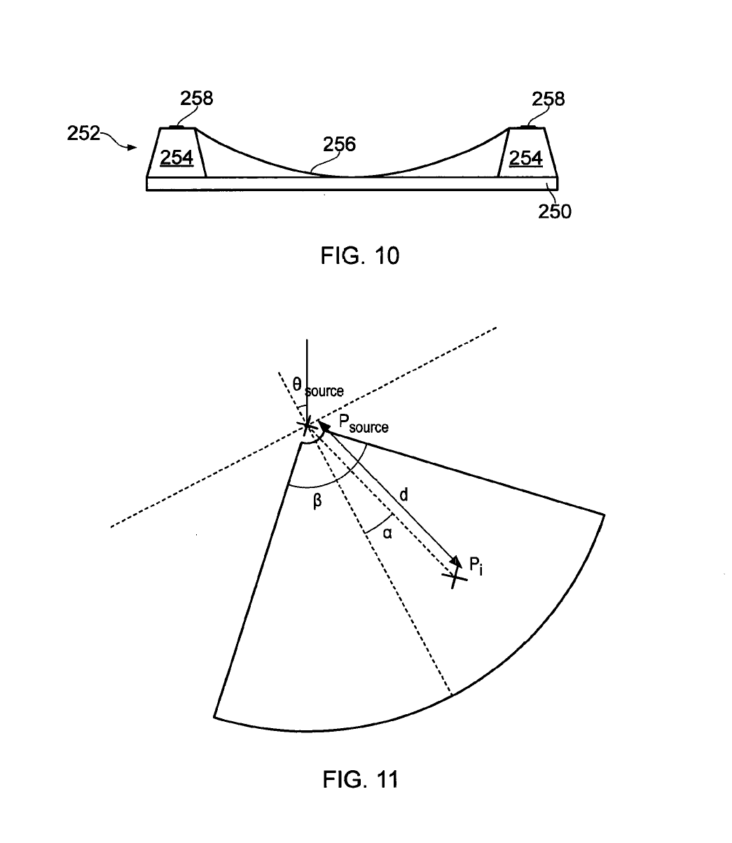

FIG. 10 illustrates the curved spacing sheet,

FIG. 11 illustrates the x-ray source parameters,

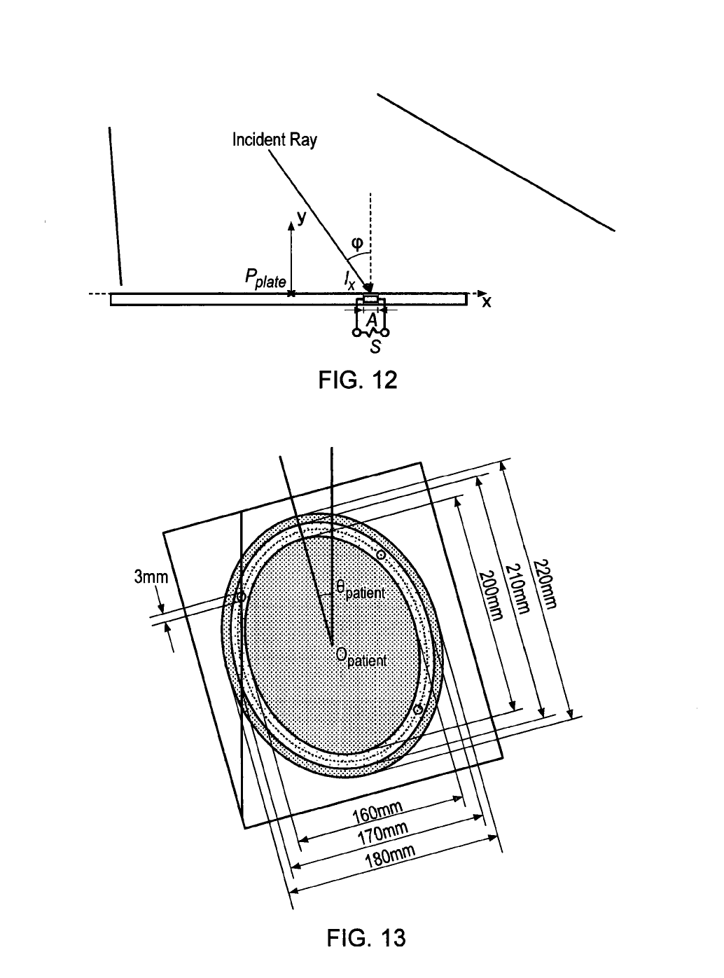

FIG. 12 illustrates the x-ray detector parameters

FIG. 13 illustrates the patient parameters,

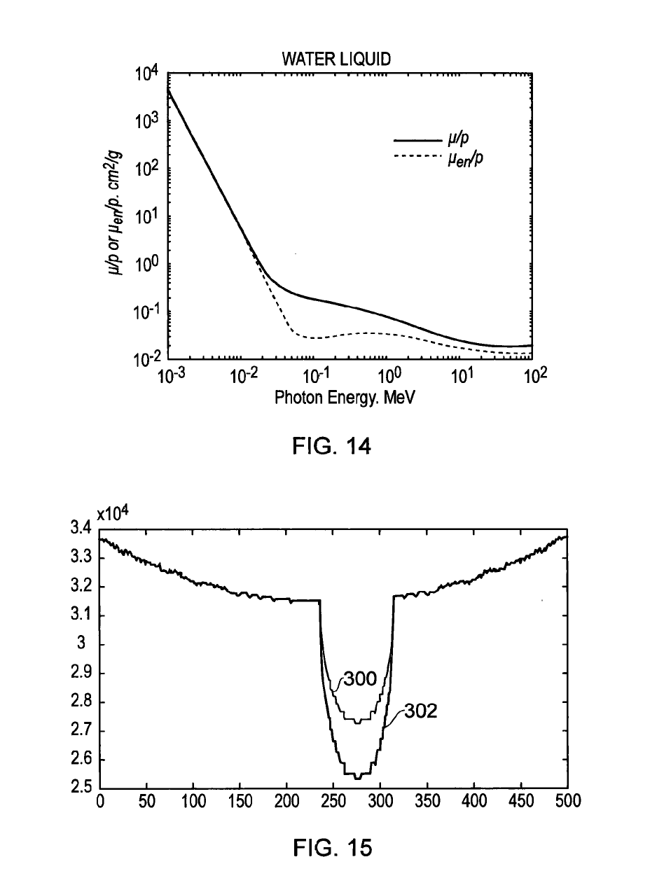

FIG. 14 shows water absorption as a function of x-ray energy,

FIG. 15 shows the shadow cast by steel or titanium fiducials, and

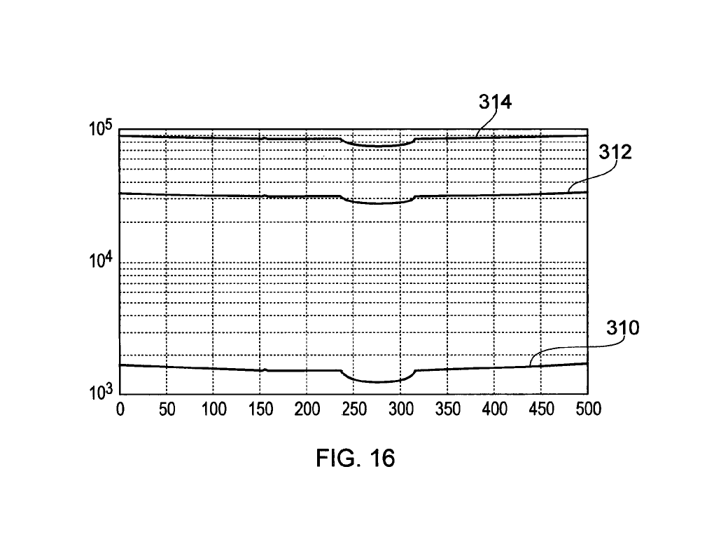

FIG. 16 shows shadow cast by a stainless steel fiducial for three different x-ray source energies.

Referring to FIG. 1, apparatus is shown that comprises a surgical robot 2.

The surgical robot 2 comprises a base 4 and a moveable arm 6. The moveable arm 6 comprises a first arm section 8, a second arm section 10 and a third arm section 12. A first rotary joint 14 links the base 4 to the first arm section 8, a second rotary joint 16 links the first arm section 8 to the second arm section 10 and a third rotary joint 18 linked the second arm section 10 to the third arm section 12. A tool holder 20 is provided at the distal end of the third arm section 12. Position encoders provided in each of the first, second and third rotary joints 14, 16 and 18 allow (after suitable calibration etc) the position of a reference point 19 on the tool holder 20 and the orientation of the tool holder 20 to be measured. In other words, the position of the reference point 19 and the orientation of the tool holder 20 are known in a local coordinate system of the surgical robot. This local coordinate system of the robot is referred to herein as the robot coordinate system. The surgical robot 2 is controlled via a computer 22. A surgical bed 24 is provided adjacent the surgical robot 2 for receiving the patient.

The apparatus of FIG. 1 also includes an x-ray source 26 that is held by the tool holder 20. A repeatable mounting mechanism is provided on the tool holder 20 and x-ray source 26 to allow the x-ray source 26 to be mounted to the tool holder 20 as and when required in a repeatable position (i.e. in a known position and orientation relative to the reference point 19). An x-ray detector 28 is mounted to the bed 24. A patient 30 is placed on the bed 24 with their head positioned on the x-ray detector 28 and adjacent the moveable arm 6 of the surgical robot 2.

As explained above, the use of the surgical robot 2 to hold the x-ray source 26 removes the need to provide separate x-ray apparatus (e.g. a CT scanner) in the operating theatre. The position and orientation of the x-ray source 26 relative to the tool holder is known (following suitable calibration of the surgical robot) and hence the position and orientation of the x-ray source 26 is known in the robot coordinate system (x,y,z).

It will now be described, with reference to FIGS. 2 and 3, how the x-ray detector 28 may be registered to the surgical robot.

Referring to FIG. 2, it is illustrated how the position and orientation of the x-ray detector 28 may be determined using the x-ray source 26 mounted to the surgical robot 2.

The x-ray detector 28 comprises a digital x-ray plate 40 of known type. The digital x-ray plate may comprise a two dimensional pixel array. Each pixel may have a size of 75 μm, although any suitable x-ray plate may be used.

A Perspex sheet 42 is secured to the front face of the digital x-ray plate 40. The outermost face of the Perspex sheet 42 carries an x-ray visible fiducial marker 44 in the vicinity of each corner of the digital x-ray plate 40. In this example, each x-ray visible fiducial marker 44 is in the form of a cross but other shapes of fiducial marker could be used. The fiducial markers 44 are thus spaced apart from the digital x-ray plate 40 by a first distance. The lateral (in-plane) separation of the markers is the second distance. Four shadows 46 are cast onto the digital x-ray plate 40 by the four x-ray visible fiducial markers 44; this provides information on the relative position and orientation of the x-ray detector 28 and the x-ray source 26.

Referring now to FIG. 3, it is shown how the x-ray detector 28 may acquire images with the x-ray source 26 placed in at least two different positions about 20-30 cm from the x-ray detector 28. More than two different positions may be used. If sufficient fiducial markers are provided, a single position may be sufficient. The position and orientation of the x-ray source 26 (i.e. all six degrees of freedom) is known in the robot coordinate system in each adopted position. This information about the position and orientation of the x-ray source 26 can be combined with the information on the relative position and orientation of the x-ray detector 28 and the x-ray source 26 provided by the shadows 46 that are cast onto the digital x-ray plate 40 for each position of the x-ray source.

After analysis of the x-ray images, the position and orientation of the x-ray detector 28 is known in the robot coordinate system. In FIGS. 2 and 3, the registration process is shown without the patient present to illustrate the principle. As explained below, the patient may actually be present during this registration. However, assuming the x-ray detector 28 can be immobilised so that it does not move when the patient is placed adjacent to it, it would also be possible to perform the x-ray detector registration process without the patient to minimise their x-ray exposure. A registration check could then be performed to confirm the x-ray detector registration is still valid.

It will now be described, with reference to FIGS. 4 to 6, how a patient may be provided with x-ray visible fiducial markers to allow registration to the surgical robot.

Referring to FIG. 4, it is shown how three bone screws 60a-60c may be secured to the skull 62 of a patient. The bone screws 60a-60c may be subcutaneously buried after attachment and they may be attached in advance of any planned surgical robot based procedure. The bones screws may even be pre-existing bone screws; e.g. they may have been implanted previously to attach a plate to the skull in a previous craniotomy. The bone screws are preferably spaced apart around the skull.

Prior to surgery but after screw implantation, a CT scan is performed on the patient. The screws act as x-ray visible fiducial markers and can be seen in the three dimensional CT image. The position of the centre of the screw head of each of the bone screws 60a-60c can be found from the pre-operative CT image and this allows the relative positions of the bone screws 60a-60c to be determined in three dimensions. The CT image can also show other anatomical structures (e.g. the skull bone etc and features within the brain). A CT angiogram (CTA) and/or MRI images may also be acquired and the position of features within such images can be determined relative to the position of the bone screws 60a-60c.

Referring to FIG. 5, a Leksell head frame 80 is shown attached to a patient 82. The attachment of a Leksell head frame to a patient is usually done on the day of surgery. As shown in FIG. 5, the x-ray detector 84 may be fixed to the frame in the anteroposterior (AP) orientation. The x-ray detector 84 can also be attached in the lateral directions (not shown). In this manner, the x-ray detector 84 can be placed in three different orientations adjacent the head to allow images to be captured from three different perspectives. Preferably, a repeatable attachment mechanism is provided to allow the x-ray detector 84 to be attached to the frame in three repeatable orientations. The x-ray detector 84 may include a digital x-ray plate and a front Perspex sheet incorporating x-ray visible fiducial markers as described above.

It should be noted that in FIG. 5 the patient has bone screws attached to their skull to act as x-ray visible fiducial markers. The fixator pins 86 of the Leksell head frame may also provide additional, or alternative, x-ray visible fiducial markers. A CT image of the patient is preferably acquired after attachment of the Leksell head frame to establish the relative positions of the fixator pins 86.

FIG. 6 shows a head clamp 100 that is an alternative to the Leksell head frame 80 described with reference to FIG. 5. The head clamp 100 comprises a first arm that carries one pin 102 and a second arm to which a pair of pins 104a and 104b are attached by a yoke. An x-ray detector 106 is affixed to the head clamp 100.

Referring to FIG. 7, a procedure for registering a patient and x-ray detector to the surgical robot will be described.

Prior to the robot based surgical procedure, a patient 120 has bone screws 122 affixed to their skull to act as x-ray visible fiducial markers as described above in more detail with reference to FIG. 4. A CT scan of the patient is then performed to establish the relative positions of the bone screws 122. An MRI or CTA scan can also be performed that shows structures or blood vessels within the brain and the position of such structures relative to the bone screws 122. A pre-operative planning process may then be performed to determine the trajectory and target position of an implantable neurosurgical device, such as a catheter or electrode.

On the day of surgery, the patient's head is immobilised in a fixed position relative to the x-ray detector 28 that has a known position in the robot coordinate system. The head immobilisation may be done, for example, using a head frame described with reference to FIG. 5 or a head clamp as described with reference to FIG. 6. The x-ray detector 28 comprises a digital x-ray plate 40 with a Perspex sheet 42 that carries four x-ray visible fiducial markers 44 as described with reference to FIGS. 2 and 3.

After the patient's head and the x-ray detector 28 have been immobilised, the moveable arm of the robot is used to place the x-ray source 26 in a first position and orientation relative to the patient. This first position is illustrated as position P1 in FIG. 7. The x-ray source 26 is arranged to emit a diverging beam of x-rays 130 that fall on the entire x-ray detector 28 and two of the three bone screws 122 affixed to the patient's skull. A first x-ray image is thus acquired.

The moveable arm of the surgical robot is then used to place the x-ray source 26 in a second position and orientation relative to the patient. This second position is illustrated as position P2 in FIG. 7. The x-ray source 26 is arranged to emit a diverging beam of x-rays 130 that again falls on the entire x-ray detector 28 and two of the three bone screws 122 affixed to the patient's skull, including at least one bone screw visible in the first x-ray image. A second x-ray image is then acquired.

Analysis of the first and second x-ray images, in particular the shadows cast by the four x-ray visible fiducial markers 44, allows the position and orientation of the x-ray detector 28 to be calculated in the robot coordinate system. The position of the bone screws 122 in the first and second images, along with the known (i.e. measured by CT) relative position of the bone screws 122, allows the position of those bone screws to also be found in the robot coordinate system. In this manner, the patient can be very quickly registered to the surgical robot. Any instruments trajectories or brain target sites identified pre-operative can also be mapped into the robot coordinate system.

Referring next to FIGS. 8-16, it will be described in more detail how to resolve plate and patient position for apparatus of the type described above.

FIG. 8 illustrates the basic geometry of the apparatus. A digital x-ray detector plate 200 is arranged to receive x-rays from an x-ray source 202. A Perspex sheet 204 is placed on the detector plate 200 to space the patient's head 206 from the plate 200. First fiducial markers 208 are implanted in the patient's head 206 and second fiducial markers 210 are provided on the uppermost surface of the Perspex sheet. Shadows cast by the first and second fiducial markers onto the plate 200 give information on fiducial marker location. The x-ray source 202 may be placed at different locations to establish the position of the fiducials in 3D space.

Referring to FIG. 9, fiducial markers 220 spaced apart from an x-ray detector plate 222 by a Perspex plate 224 are shown. The spatial resolution with which the position of the fiducial markers can be established will now be described for a number of different scenarios.

In a first example, it is assumed the Perspex plate 204 is 10 mm thick, the robot arm locates the x-ray source 202 a nominal 300 mm away from the Perspex, the robot arm is positioned over the centre of one of the markers on the Perspex, the markers cast a perfect shadow on the plate, the x-ray detector plate has pixel size of 0.1 mm and that the signal processing algorithms can detect a 0.5 pixel shift in the shadow.

The smallest resolvable movement (d_{resolvable}) is then given by the equation:

$${d}_{resolvable} = \frac{Δ p ( {d}_{source} - {t}_{perspex} )}{2 {t}_{perspex}} \qquad ( 1 )$$

where the Δp is the pixel size, d_{source} is the distance of the x-ray source from the x-ray detector plate 222 and tₚₑᵣₛₚₑₓ is the thickness of the Perspex plate 224. In this first example, d_{resolvable} is 1.5 mm.

This resolution may be sufficient for certain applications, but can be improved in a number of different ways. For example, it would be possible to increase the resolution of the x-ray detector plate 222, reduce the distance of the robot from the plate (which may require that the patient is not in position), increase the distance of the fiducial markers 220 from the x-ray detector plate 222 and/or interpolate the position over multiple (e.g. four) different markers.

In a second example, the patient is not present during the process which allows the plate to be placed 100 mm from the x-ray source. The fiducial markers 220 are also placed 20 mm away from the x-ray detector plate 222. A reduced pixel size of 0.075 mm is also used. This leads, using equation (1), to a smallest resolvable movement (d_{resolvable}) of 0.19 mm which is more than sufficient for most guided neurosurgical procedures.

In a third example, the patient is present so the plate is placed 250 mm from the x-ray source. The fiducial markers 220 are again placed 20 mm away from the x-ray detector plate 222. A reduced pixel size of 0.075 mm is also used. This leads, again using equation (1), to a smallest resolvable movement (d_{resolvable}) of 0.49 mm which is sufficient for most guided neurosurgical procedures, although other effects (e.g. noise, artefacts etc) may mean this might be insufficient for the highest accuracy procedures.

Referring to FIG. 10, an x-ray detector plate 250 with a concave Perspex plate 252 is shown. The Perspex plate 252 has edge regions 254 that are thicker than a central concave region 256 for receiving a patient's head. Fiducial markers 258 are located on the thicker edge regions 254. In the example of FIG. 10, the x-ray source may be placed 300 mm away from the plate 252 and the fiducial markers 258 placed 40 mm from plate 250. Assuming a pixel size of 0.1 mm and using equation (1), the smallest resolvable movement (d_{resolvable}) is 0.325 mm.

A similar approach can be used to resolve patient position. The assumptions are made that both the location of the plate and the arm of the robot holding the source are known and that the fiducials attached to the patient are located somewhere between the source and the plate. The fiducials attached to the patient can be assumed to have a nominal 3 mm diameter.

The resolvable lateral resolution of the patient fiducials is then given by equation (2), namely:

$${d}_{resolvable} = \frac{Δ p ( {d}_{robot} - {d}_{fiducial} )}{2 {d}_{fiducial}} \qquad ( 2 )$$

where d_{robot} is the spacing between the robot arm and the x-ray detector plate and d_{fiducial} is the spacing between the fiducial and the detector plate. It assumed that the robot arm is 300 mm from the plate, the robot arm is positioned over the centre of one of the patient fiducials, the fiducials cast a perfect shadow on the plate, the plate has pixel size of 0.1 mm and the algorithms will detect a 0.5 pixel shift in the shadow. A fiducial at a distance of 100 mm from the x-ray detector plate can be resolved with a lateral resolution of 0.075 mm. A fiducial at a distance of 200 mm from the x-ray detector plate can be resolved with a lateral resolution of 0.025 mm

Due to the conic nature of the beam, the fiducial image is magnified depending on its longitudinal location in the beam. This may be used to locate the approximate 3D location of the fiducial using just one image.

The magnification (M) is given by:

M=d_{source}/(d_{source}−d_{fid}) (3)

Application of the reciprocal rule means the change in magnification with longitudinal movement is given by:

$$\frac{\partial M}{\partial {d}_{fid}} = \frac{{d}_{source}}{{( {d}_{source} - {d}_{fid} )}^{2}} \qquad ( 4 )$$

The change in projection diameter with longitudinal movement is therefore:

$$\frac{\partial {D}_{proj}}{\partial {d}_{fid}} = \frac{{D}_{fid} {d}_{source}}{{( {d}_{source} - {d}_{fid} )}^{2}} \qquad ( 5 )$$

Assuming a detectable change is ½ pixel on each side of the fiducial, which equals a 1 pixel change in projection shadow diameter, then:

$$Δ {d}_{fid} = \frac{0.1 {( {d}_{source} - {d}_{fid} )}^{2}}{{D}_{fid} {d}_{source}} \qquad ( 6 )$$

It is also assumed that the robot arm is 300 mm from the plate, the robot arm is positioned over the centre of one of the patient fiducials, the fiducials cast a perfect shadow on the plate, the plate has pixel size of 0.1 mm, the algorithms will detect a 0.5 pixel shift in the shadow, the fiducials are between 100 and 200 mm from the plate and the fiducial is 3 mm in size. For a fiducial nominally located 100 mm from the detector plate, the longitudinal position can be established to within 4.44 mm. For a fiducial nominally located 200 mm from the detector plate, the longitudinal position can be established to within 1.1 mm. Longitudinal position can thus be calculated, albeit to relatively low accuracy, to allow the robot to move to an optimal position to refine the fiducial location.

Referring to FIGS. 11 to 16, a mathematical model describing operation of the above described technique will be described.

FIG. 11 illustrates the x-ray source model. It is assumed that the source is a point source, has constant intensity over its beam width and is monochromatic. The source has a beam width (β), α is the Scalar angle of the point, P_{source} is the location of the source, d is the distance of the point from the source, θ_{source} is the source angle and Iₚᵢ is the intensity of the source at Pᵢ. It can thus be assumed that:

$$I ∝ \frac{1}{{d}^{2}} F ( α ) \qquad ( 7 )$$

where F(α)=1 when −β/2<α<β/2 or F(α)=0 otherwise.

FIG. 12 illustrates the plate model. In particular, S is the output signal of sensor, L is the length of sensor pixel, P plate is the location plate, φ is the angle of incidence and I is the intensity at x,y. The sensor output is thus given by:

S∝∫_{x-L/2}^{x+L/2}Iₓ cos(φₓ)dx (8)

where φ=α+θ.

The patient is modelled as a series of ellipses representing the head, skull and brain regions. The patient implanted fiducials are represented as 3 mm metal balls for convenience. Different substances have different Hounsfield units (HUs) as illustrated in table 1.

TABLE 1
Assumed material properties

| Substance | HU |
|---|---|
| Air | −1000 |
| Fat | −100 to −50 |
| Water | 0 |
| CSF | 15 |
| Blood | +30 to +45 |
| Muscle | +10 to +40 |
| Grey matter | +37 to +45 |
| White matter | +20 to +30 |
| Soft Tissue, Contrast | +100 to +300 |
| Bone | +1000 |
| stainless steel | 2,222 ± 737 HU |
| titanium | 2,921 ± 218 HU |

Referring to FIG. 13, a patient model is illustrated where skin is given a value of 100 HU, brain 30 HU, skull 1000 HU and the fiducials are either 2000 or 3000 HU (i.e. stainless steel or titanium).

FIG. 14 shows the absorption coefficient, μ, of water (ρ=1 g/cm3). Using this and the HU for other materials the approximate absorption coefficients for skin, bone, brain and fiducials are calculated and used in the model.

Referring to FIG. 15, the patient model was placed with its origin 120 mm above a plate of element size 0.1 mm and width 5 cm. A single fiducial ball of 3 mm diameter was placed in a position (1, 65) within the patient's head. The source was placed at the position (0, 300) and pointed down towards the plate. Two runs were performed to compare the shadow cast by balls of 2000 HU (e.g. Stainless Steel) and 3000 HU (e.g. Titanium) at a source energy of 45 kev. As shown in FIG. 15, both stainless steel (curve 300) and titanium (curve 302) produce a detectable shadow.

FIG. 16 shows a comparison of different source energies of 30 kev (plot 310), 45 key (plot 312) and 60 kev (plot 314) with a fiducial ball of 2000 HU. The lower energies are absorbed more by the head and skull, but the relative absorption of the fiducial ball is also greater.

It can thus be seen that patient position is resolvable to acceptable accuracy. Preferably, two or more images are taken per fiducial at different angles. Three fiducials are preferred to fix the patient position in 3D space, and it is preferred that at least six X-ray images are taken to register the patient. However, some scans may pick up more than one fiducial marker thereby reducing the total number of X-rays required. The x-rays can be of very low dosage, especially when compared to an O-arm. The above concept thus offers a convenient technique for patient robot registration. Further accuracy and/or mathematical simplification can be obtained if the relative locations of the patient mounted fiducials are known (e.g. pre-measured) and taken as constants in the analysis.

The skilled person would appreciate the various alternative procedures and apparatus that could be used to implement the invention. Although a human patient is described above, the technique could be applied to any animal or human subject.

1. Surgical apparatus comprising;
a surgical robot having a moveable arm for carrying a surgical tool, and
x-ray imaging apparatus including at least one of an x-ray source and an x-ray detector,
wherein the moveable arm of the surgical robot is configured to carry at least one of the x-ray source and the x-ray detector.

2. An apparatus according to claim 1, wherein the x-ray imaging apparatus comprises an x-ray source and the moveable arm is configured to carry the x-ray source.

3. An apparatus according to claim 2, wherein, during use, the moveable arm is configured to move the x-ray source into a plurality of positions relative to the subject to allow a plurality of x-ray images to be acquired from a plurality of different perspectives.

4. An apparatus according to claim 1, wherein the surgical robot comprises a releasable attachment mechanism that allows at least one of the x-ray source and the x-ray detector to be attached to the moveable arm when x-ray imaging is to be performed.

5. An apparatus according to claim 4, wherein a tool holder is provided at the distal end of the moveable arm for retaining a surgical tool in a repeatable position, wherein the tool holder also provides the releasable attachment mechanism for attaching at least one of the x-ray source and the x-ray detector to the moveable arm.

6. An apparatus according to claim 1, wherein the x-ray imaging apparatus includes at least one x-ray detector comprising a digital x-ray plate.

7. An apparatus according to claim 6, wherein the at least one x-ray detector comprises a fiducial marker unit located adjacent the digital x-ray plate, the fiducial marker unit comprising a plurality of x-ray visible fiducial markers having a known position relative to the digital x-ray plate.

8. An apparatus according to claim 1, comprising a frame for attachment to a subject, wherein the x-ray imaging apparatus includes at least one x-ray detector that is attachable to the frame.

9. An apparatus according to claim 1, wherein the moveable arm comprises a plurality of motorised joints and a plurality of position encoders for measuring the position of a reference point at the distal end of the moveable arm in a coordinate system of the surgical robot.

10. An apparatus according to claim 1, comprising an analyser for analysing one or more x-ray images acquired by the x-ray imaging apparatus, wherein the apparatus includes one or more x-ray visible fiducial markers and the analyser is arranged to analyse the one or more x-ray images to determine the position of the x-ray visible fiducial markers in a coordinate system of the surgical robot.

11. An apparatus according to claim 10, wherein the one or more x-ray visible fiducial markers comprise a plurality of bone attachable fiducial markers for direct attachment to a bone of a subject.

12. An apparatus according to claim 10, comprising a head clamp for attachment to the head of a subject, wherein the one or more x-ray visible fiducial markers comprise a plurality of x-ray visible fiducial markers attached to the head clamp.

13. An apparatus according to claim 10, wherein at least some of the plurality of x-ray visible fiducial markers have a predetermined positional relationship.

14. An apparatus according claim 1, wherein the surgical robot comprises a neurosurgical robot.

15. A kit for adapting a surgical robot to perform x-ray imaging, the surgical robot comprising a moveable arm for carrying a surgical tool, wherein the kit comprises an x-ray source and at least one x-ray detector, wherein the x-ray source or the x-ray detector is adapted to be carried by the moveable arm.
